(19) United States
(12) Patent Application Publication
(10) Pub. No.: US 2013/0090521 A1
(43) Pub. Date: Apr. 11, 2013

(54) TIMED-RELEASE TENSIONED OR COMPRESSED FIBERS

(71) Applicant: Novo Contour, Inc., Edmonds, WA (US)

(72) Inventors: Michael P.H. Lau, Edmonds, WA (US); Leonard Pease, Bountiful, UT (US)

(73) Assignee: NOVO CONTOUR, INC., Edmonds, WA (US)

(21) Appl. No.: 13/648,188

(22) Filed: Oct. 9, 2012

Related U.S. Application Data
(60) Provisional application No. 61/545,002, filed on Oct. 7, 2011.

(51) Int. Cl. A61F 2/02; A61B 17/04

(52) U.S. Cl. 600/30; 606/230; 600/37

(57) ABSTRACT

A timed-release tensioned core-shell fiber comprises a core positioned within a shell. The shell is configured to hold the core under said tension or compression. The shell is at least partially removable and releases at least a portion of the tension or compression of the core in response to the shell being removed. The shell is at least partially removable by erosion or degradation due to mechanical, chemical, electrical, physical, or thermal processes, or combinations thereof. In some embodiments, the erosion or degradation of the shell may include biodegradation, bioerosion, photooxidation, or photodegradation. A fiber mesh comprised of core-shell fibers may be tuned for timed release of contraction or expansion forces in response to timed release of tension or compression of the core. The fiber mesh may be used in a medical device, bandage, implant, tissue construct, or sling. A suture may also comprise a core-shell fiber.

### CROSS-REFERENCE TO RELATED APPLICATION

This application claims the benefit of U.S. Provisional Patent Application No. 61/545,002, filed Oct. 7, 2011, the disclosure of which is incorporated by reference herein in its entirety.

### TECHNICAL FIELD

The present application relates to fibers and medical products including meshes, slings, bandages, sutures, tissue scaffolds, and the like, formed from these fibers.

### BACKGROUND

Urinary incontinence and pelvic floor disorders adversely affect millions of women leading to social embarrassment, incapacitating falls, and nursing home admission. Genital prolapse, including cystocele, rectocele, enterocele, and uterine prolapse, along with stress incontinence, affect nearly one in four U.S. women (28.1 million), and by 2050, the number is projected to grow to nearly 44 million.

Women, especially elderly women, often find this pelvic floor disorder too embarrassing to disclose. However, the effect of genital prolapse can be quite disabling. The prolapse of the vaginal wall and pelvic organs are due to the weakening of the endopelvic fascia and supportive ligaments of the pelvis. The weakness is usually caused by childbirth and is compounded by the aging process and occasionally surgical trauma.

Surgical treatment for urinary incontinence involves the placement of either natural tissue or synthetic mesh in a patient to support the urethrovesicle junction (UVJ), commonly called the bladder neck. In healthy patients, the UVJ is adequately supported by a thin (˜3 mm thick) layer of pelvic fascia. When the fascia weakens or elongates over time or stretches due to childbirth, the UVJ falls from its preferred position superior to the base of the bladder during Valsalva's events, allowing urine loss. To provide additional support, most gynecological surgeons use polymer meshes that are readily available, sterilized, and inserted as a suburethral sling on an outpatient basis.

The repair of a prolapse, in principle, usually involves the plication of the supportive endopelvic fascia or ligaments, using sutures, after extensive dissection of the pelvic structures along the tissue planes. In many women, especially the elderly, the supportive endopelvic fascia or ligaments are so thinned out or non-existent as to make suturing to plicate very challenging, if not impossible. Traditionally, autologous or donor tissue is used to supplement the deficient pelvic support tissue. Such use of biologic materials adds significant complexity and extensiveness of the surgery and has its associated risks. For these reasons, more surgeons are turning to the use of synthetic surgical mesh or acellular collagen matrix of porcine dermal material to augment the traditional genital prolapse repair. These materials have been increasingly included as part of commercially pre-packaged minimally invasive surgical kits for pelvic floor repair. The kits also usually involve anchoring the mesh material to some fixed tissue points in the pelvis or relying on tissue-mesh friction to hold the mesh in place. However, properly tensioning commercially available mesh is challenging with state-of-the-art surgical techniques and material. Historically, treatment of incontinence and pelvic organ prolapse called for open surgery. Surgical trainees learned how to properly tension sutures and tissue to provide optimal benefit. Over-tensioning may lead to urethral stenosis, voiding dysfunction, urinary retention, and increased risk of mesh erosion, while under-tensioning may render the surgery ineffective. Success rates for treatment of stress urinary incontinence (SUI) by open surgery, such as retropubic urethropexy, can exceed 97%, but open surgery requires several weeks of recovery. With the advent of minimally invasive surgical techniques, patient recovery is more rapid, but long-term success rates have been much more modest and disparate, ranging from less than 70% to 85%, in part because achieving optimal levels of tension is challenging in confined environments. Recent studies show more than 30% of patients require two surgeries to correct incontinence. Indeed, successful treatment often depends significantly on an individual surgeon's technique, leading to dramatic differences in success rates among clinics. The inability to readily achieve optimal levels of tension in confined spaces is a general problem affecting nearly all pelvic reconstructive surgeries.

An ideal mesh should be such that it would not require over-tensioning during its surgical deployment and yet would apply an optimal amount of tension that will increase over time, in situ, to gradually contract the tissue to achieve the surgical objectives. As a temporary scaffolding, an ideal mesh will eventually thin and weaken as the fibers biodegrade without a long-term trace. Such a surgical technique allows the surgeon to place the mesh without the need to over-tension or over-dissect, while implementing more flexible minimally-invasive surgical access techniques. Current meshes, in contrast, only apply a fixed amount of tension. Ideal meshes also biodegrade to prevent erosion of adjacent tissues after they let new tissue grow through the temporary scaffolding to allow the target endopelvic fascia strengthen sufficiently to support the UVJ and the vaginal wall without assistance (typically 3-4 months). For the current meshes, nearly 70% of erosion cases occur more than 1 year post surgery when a biodegradable mesh would have fully degraded.

The long standing need for the subject matter of this disclosure was recently elevated by two recent FDA warnings, one late in October of 2008 and the other in July of 2011. Despite their broad clinical acceptance, synthetic mesh for the treatment of pelvic floor disorders including urinary incontinence and pelvic organ prolapse is associated with erosion and/or contraction. The erosion complication occurs when the polymer mesh cuts through (i.e., erodes) adjacent tissue, penetrating the bladder, vagina, or urethra depending on initial placement. The eroded area causes loss of organ function and chronic discharge, becomes susceptible to infection, often causes painful rejection of the mesh, and requires surgical reintervention. In 1999, the FDA removed the worst offending meshes completely from the market. Since that time, polypropylene meshes have captured the greatest share of the market, though they also have significant erosion rates leaving the long standing need firmly in place. The contraction complication occurs when the implant stimulates the formation of collagen along with the infiltration of fibroblasts and/or myofibroblasts. For example, the mere presence of some polymers or the release of their degradation products may induce a foreign body response that leads to formation of thick avascular collagenous shells that contract with the ratcheting of fibroblasts and/or myofibroblasts. Alternatively, the contraction may be associated with the normal wound healing process. In either case, the induced tension associated with contraction and fibrosis leads to complications for pelvic floor surgeries that may be avoidable or minimized with the present disclosure.

In some instances there are needs for both tensioning and expansion. However, these needs generally occur on different time scales, allowing for engineering design to optimize the response. For example, tensioning may be required earlier in the healing response before collagen formation occurs in sincerity (e.g., less than 10-20 days), whereas expansion may be required at a later time to oppose the ratcheting of the fibroblasts and myofibroblasts (e.g., around 10-180 days). For all of these needs, many of which are long standing, the present disclosure provides solutions in many forms, including suture and mesh form, though one skilled in the art will understand significant variations, combinations, and permutations thereon.

### SUMMARY

This application relates to timed-release tensioned or compressed fibers and medical products including meshes, slings, bandages, sutures, tissue scaffolds, and the like, formed from these fibers that are capable of gradually and tunably applying contraction and/or expansion forces to tissues in vivo.

In various embodiments, a timed-release tensioned core-shell fiber comprises a core under tension or compression. The core is positioned within a shell and the shell is configured to hold the core under said tension or compression. The shell is at least partially removable and configured to release at least a portion of the tension or compression of the core in response to the shell being at least partially removed.

In various embodiments of the core-shell fiber, the shell is at least partially removable by erosion or degradation of the shell. The erosion or degration may be accomplished using mechanical, chemical, electrical, physical, or thermal processes, or combinations thereof. For example, the erosion or degradation of the shell may include at least one of biodegradation, bioerosion, photooxidation, or photodegradation.

The shell may comprise a surface-eroding polymer or a bulk-eroding polymer. In various embodiments, the erosion or degradation of the shell is controllable.

This summary above is provided to introduce a selection of concepts in a simplified form that are further described below in the Detailed Description. It should be understood that this summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic diagram illustrating a combination of core-shell fibers on a rigid interdigitating structure to effect expansion by core-shell fiber shrinkage;

FIG. 2 is a pictorial diagram illustrating a pretensioned core-shell fiber fabrication and degradation scheme;

FIG. 3 is a pictorial diagram illustrating (a) a coordinate system and stresses relative to a fiber for a base case (denoted by ^{o}) and relaxed case (denoted by ′); and (b) mesh configurations for lifting the UVJ (circle), wherein the mass of the UVJ and corresponding tissue is mₜ and the distance between ends of the mesh is lₐ;

FIG. 4 is a pictorial diagram illustrating a diagram of tip and internal delamination, showing that only tip delamination creates a stress-free region to derive delamination; FIG. 5 is a graph illustrating Gₛₛ/E_{c}H versus u_{zz}^{o}, for typical values reported in the results and discussion section;

FIG. 6 is a set of graphs illustrating (a) ũ_{z}(\tilde{r},1) versus \tilde{r} for u_{zz}^{o}=0.01, 0.5, 1, 2, and 10 and \tilde{h}=0.5; (b) deformation at the fiber center, ũ_{z}(0,1), core-shell interface, ũ_{z}(\tilde{h},1), and exterior shell surface, ũ_{z}(1,1), versus \tilde{h}; (c) ratio of the deformation at the core-shell interface, ũ_{Z}(\tilde{h},1), to the maximal core-shell interfacial deformation versus hₛ/h_{c} for u_{zz}^{o}=0.01, 1, 2, and 10; (d) ũ_{z}(\tilde{h},1) versus E_{c}/Eₛ for H/L=0.01, 0.1, and 1; (e) ũ_{z}(\tilde{h},1) versus v_{c} for vₛ=0.25, 0.33, 0.45, and 0.495; and (f) ũ_{z}(\tilde{h},1) versus E_{c}/Eₛ for v_{c}=0.25, 0.33, 0.45, and 0.495;

FIG. 7 is a set of graphs illustrating (a) u_{zz}* versus (γₛ+γ_{c}−γ_{sc})/(E_{c}H) for h/H=0.01, 0.25, 0.5 and 0.99 (note each curve goes negative at sufficiently low values of (γₛ+γ_{c}−γ_{sc}/(E_{c}H)); (b) u_{zz}* versus h/H for (γₛ+γ_{c}γ_{sc})/(E_{c}H)=0.1, 0.01, 0.001, and 10; (c) u_{zz}* versus E_{c}/Eₛ for H/L=0.01, 0.1, and 1; and (d) u_{zz}* versus v_{c} for vₛ=0.25, 0.33, 0.45, and 0.495; and

FIG. 8 is a set of graphs illustrating (a) Δlᵥ/lₘ^{co} versus mₜg/(2πNE_{c}h²) for (lₐ/lₘ^{co})²=0.01, 0.5, 0.9, and 0.99; (b) Δlᵥ/lₘ^{co} versus u_{zz}^{o} for E_{c}/Eₛ=0.01, 0.1, 1, and 10; (c) Δlᵥ/lₘ^{co} versus h/H for H/L=0.0001, 0.01, and 1; and (d) Δlᵥ/lₘ^{co} versus v_{c} for vₛ=0.25, 0.33, 0.45, and 0.495.

### DETAILED DESCRIPTION

The present application discloses fibers with mechanically and temporally tunable properties. Each fiber comprises a core and a shell. The quintessential feature of the fibers is that the core possesses a distinct degree of mechanical tension or compression relative to the core. If the core is in tension relative to the shell, then upon removal of the shell the core will contract. If the fiber is anchored, the removal of the shell will cause the distance between the anchor points to decrease. If the anchor points are fixed and the fiber is weight bearing, the fiber will lift the weight. Alternatively, if the core is in compression relative to the shell, then upon removal of the shell the core will expand. If the fiber is anchored and the core is somewhat rigid, the distance between the anchor points will increase. If the anchor points are fixed and the fiber is weight bearing, the fiber will lower the weight. In each case, removal of the shell releases the stored mechanical energy that can then act on the adjacent tissue. By tuning the fiber material properties, the release rate and rate of mechanical effect of the fiber can be controlled. The removal rate of the shell governs the rate of mechanical energy release and the temperospatial profile of the fiber core.

The disclosed specifications herein describe various compositions of these fibers, criteria for tuning their function, methods for their manufacture, and their incorporation into useful medical devices. This disclosure presents five exemplary ways and combinations thereof to construct or fabricate the above fibers, without limiting the spirit and scope of the invention. Those skilled in the art will recognize additional means or methods of achieving the fibers, which also remain within the scope and spirit of the invention.

First, the core may be placed in tension by purely mechanical means. For example, the core of the fiber may be stretched to a preferred length or to a preferred tension by an external mechanical force. Specifically, a core-only fiber may be clamped at its ends, and increasing the distance between the clamps applies a tension to the core. The tension may be fixed in place by securing the core with a shell. The ends may be specifically annealed or affixed by a variety of means (e.g., tying, clamping, crimping, etc.) to the shell to prevent delamination. Although shear forces between the core and the shell will cause/allow both to contract, tension remaining in the core remains available to act on adjacent tissue after removal of the shell.

Second, the core may be placed in compression by means of swelling it. For example, the core may be comprised of a dry-formed hydrogel. A non-swelling or minimally swelling shell may be applied to the dry hydrogel. Upon implantation in vivo or exposure to hydrating solutions such as water, the core will swell, at least partially, building up compression within the core as the shell resists the expansion due to the swelling. This is a means of placing the shell in tension. Removal of the shell will release the compression, allowing the core to further swell and expand to oppose tissue contraction or extend the length of adjacent tissue. In this example, a range of hydrogel compositions are viable from simple uncharged hydrogels to polyelectrolyte hydrogels, inter alia.

In a further example of this, the core comprises a series of hydrogel rods having a central string or strand connecting the hydrogel rods. In some embodiments, the core also comprises relatively stiff (higher elastic modulus) rods within the hydrogel rod to increase the composite stiffness of the hydrogel rod. As above, a non-swelling or minimally swelling shell is applied to each composite hydrogel rod. Upon removal of the shell, the hydrogel cores will expand. The composite cores will have enhanced mechanical strength with which to oppose compression of the adjacent tissue.

Third, the core may be placed under compression or expansion by means of thermal expansion or contraction. For example, the core may be placed under tension by first cooling it by thermal means including, but not limited to, refrigeration or freezing (e.g., by exposure to liquid nitrogen). While the core is still cool, a stress-free shell is applied. When the composite fiber temperature is raised to ambient room or body temperature, the core will ideally return to its stress-free state, while the shell will have expanded considerably. Shear forces between the core and the shell will place the core in tension and the shell in contraction. Selective removal of the shell will free the tension of the core to act on the adjacent tissue.

Similarly, the core may be placed under tension by first heating it by thermal means including, but not limited to, placement in furnaces, near heat reservoirs, exposure to thermal radiation or warm convective fluid, etc. Heating below the melting temperature and/or the glass transition temperature is preferred. While the core is still warm, a stress-free shell is applied. When the composite fiber temperature is lowered to ambient room or body temperature, the core will ideally return to its stress-free state, while the shell will have contracted considerably. Shear forces between the core and the shell will place the shell in tension and the core in contraction. Selective removal of the shell will free the compression of the core to act on adjacent tissue.

Similarly, the core may be placed under tension or compression by first heating it by thermal means including, but not limited to, placement in furnaces, near heat reservoirs, exposure to thermal radiation or warm convective fluid, etc. Heating near or above the glass transition temperature but not significantly above the melting temperature will allow the core to thermally relax. While warm, a stress-free shell is applied. When the composite fiber temperature is lowered to ambient room or body temperature, the tension or compression of the core relative to the shell will depend on the coefficients of thermal expansion of the core and shell materials. If the shell possesses a coefficient of thermal expansion greater than that of the core, then the core will be placed under compression. If the shell possesses a coefficient of thermal expansion lower than that of the core, then the core will be placed under tension. In either case, selective removal of the shell will free the compression of the core to act on adjacent tissue.

Similarly, the core may be placed under tension or compression by first cooling it by thermal means including, but not limited to, refrigeration or freezing (e.g., by exposure to liquid nitrogen). Temperatures above the glass transition temperature of the core are preferred to allow the core-only fiber to thermally relax. While the core is still cool, a stress-free shell is applied. When the composite fiber temperature is raised to ambient room or body temperature, the tension or compression of the core relative to the shell will depend on the coefficients of thermal expansion of the core and shell materials. If the shell possesses a coefficient of thermal expansion greater than that of the core, then the core will be placed under tension. If the shell possesses a coefficient of thermal expansion lower than that of the core, then the core will be placed under compression. In either case, selective removal of the shell will free the tension or compression of the core to act on adjacent tissue.

In these examples, greater differences in the coefficients of thermal expansion between core and shell are preferred. Polymeric materials are preferred for these applications because they often have relatively large coefficients of thermal expansions relative to other classes of materials.

Fourth, the core may be placed under compression by beginning with a hollow elastomeric core upon which a shell is fixed. One end of the core is capped while the other is attached to a pressure-producing device including, but not limited to, a pressurized air cylinder, air pump, compressor, liquid pump, etc. Fluid enters the core and hydrostatic pressure leads to at least partial expansion, restrained at least partially by the shell. The pressure end of the core is then cauterized or cleaved without loss of seal and then more completely sealed if necessary. In this manner, the core is placed under compression, whereas the shell is under tension. Selective removal of the shell frees the compression of the core to act on adjacent tissue.

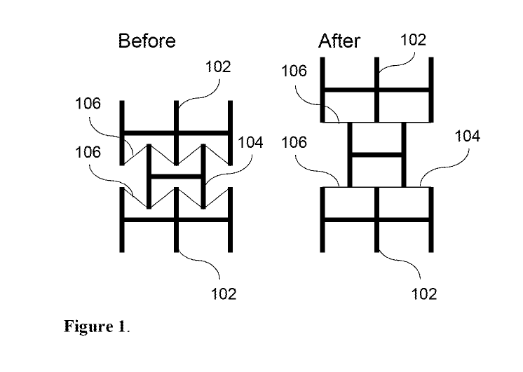

Fifth, as illustrated in FIG. 1, the contracting fibers may be placed on a rigid non-contracting, non-expanding frame. The frame has interdigitating elements 102 and 104 (with adjacent frame elements) that are connected by a core-shell fiber 106 of one length. As the shell of the fiber 106 degrades, the fiber length decreases pushing the interdigitated elements 102 and 104 apart to expand the net dimensions of the composite structure.

Notably, if the core is in tension and the shell is in compression, degrading the core first provides a means of expansion, while if the core is in compression and the shell in tension, the fiber will contract as the core selectively erodes.

Combinations of the above formulations and preparation (i.e., thermal, swelling, and mechanical) are also feasible in all their varieties. For example, a core-only fiber may be clamped, stretched, and cooled prior to application of the shell, such that upon warming to room or body ambient temperature, the core will be placed in tension. The combination allows enhanced tension not readily achievable without the combination. Similarly, a core-only fiber comprising a dry hydrogel may be heated and while at temperature be coated with a stress-free shell. Upon cooling and exposure to solvent, the core will be placed in compression. Alternatively, combinatoric formations and combinations not specifically enumerated herein lie within the scope of the invention.

Some applications may call for multiple levels of timed tension or compression or combinations thereof. Multiple levels of tension can be achieved by placing the core at a first level of tension or stretching to a first length. A first shell is applied. The core and shell are then stretched to a second level of tension or stretched to a second length, where the second length is greater than the first length. A second shell is applied. Successive shells at successive tensions or length may be applied. In another embodiment, a continuous or small stepped gradient of shells may be applied at a continuous or small stepped gradient of lengths or tensions.

Similarly, a first dry hydrogel may comprise the core of a composite fiber. A shell of a second dry hydrogel material may be applied as a shell to the first, wherein the swelling expansion in aqueous media of the first hydrogel is greater than that of the second hydrogel. Successive hydrogel shells may be applied in like manner. Finally, a non-swelling or minimally swelling coating or shell is applied. When hydrated, the core will be under the greatest compression followed by the first internal shell, second internal shell, and so forth. Selective removal of each successive shell will act on adjacent tissue as discussed above in successive fashion.

Successive shells, each with greater or less compression or tension, may be applied in varieties and combinations of the above four methods and permutations and combinations thereof.

Core-shell fibers that apply both tension and compression at respective times also lie within the scope of the invention. In a preferred embodiment, a hydrogel core is encased in a non-swelling or minimally swelling shell. The core-shell fiber is stretched to a preferred length or to a preferred tension. A second stress-free shell is applied. The tension is released such that the first shell is in tension while the second or outer shell is in compression. Selective removal of the outer shell layer releases the tension stored in the core-inner shell. Subsequent selective removal of the inner shell with hydration of the hydrogel releases the compression stored in the core. In at least one preferred embodiment, the core is placed under compression by thermal processing as discussed above and fixed with a shell at a first preferred temperature. Then the core-shell fibers are placed in tension by thermal processing at a second preferred temperature. The tension is fixed in place by another stress-free shell. At ambient room or body temperature, the core is under compression while the first inner shell is under tension. Selective removal of the outer shell releases tension to the tissue, while selective removal of the inner shell releases the desired compression. Similar multiple layer constructs to achieve successive levels of tension or compression lie within the spirit and scope of the invention.

As mentioned previously, an important feature of the fibers disclosed herein is the ability to tune the rate at which they release the tension or compression forces stored within their cores (and inner shells, where present). The external shell primarily, if not exclusively, governs the tension or compression release rate. To release the stored mechanical energy, the shell is designed to degrade, biodegrade, bioerode, photooxidize, photodegrade, or otherwise oxidize or erode to release the tension or contraction in a controlled manner. Upon release of the tension, the fiber contracts or expands by a predetermined amount, contracting the attached or adjacent target endopelvic fascia or expanding the attached or adjacent collagenous fibers along with it. Tunable erosion or biodegradation of polymer fibers is important to a well-controlled mechanical energy release rate.

Biodegrading polymers come in two varieties: bulk-eroding polymers in which polymer erosion or degradation occurs simultaneously throughout the entire fiber (i.e., both bulk and surface), and surface-eroding polymers in which only the exterior surface of the polymer undergoes erosion or degradation, leaving the center intact. In at least one preferred embodiment, the shell is composed of a bulk-eroding polymer. Here the rate of release of mechanical energy is governed, at least in part, by the local molecular weight of the polymer. At early times, the molecular weight of the polymer is high, leading to substantial values of the elastic modulus. The elastic modulus of the shell should be at least of the same order of magnitude as that of the core. As the shell polymer bulk erodes, the polymer molecular weight decreases, leading to successively lower values of the elastic modulus until the shell is no longer able to restrain the expansion or contraction of the core and the mechanical energy stored therein is released. Exemplary bulk-eroding polymers include polyesters (as defined by the presence of ester bonds) including, but not limited to, poly lactic acid, poly glycolic acid, poly(L-lactic acid), poly(D-lactic acid), poly(DL-lactic acid), and combinations thereof, etc. In a preferred embodiment, poly(lactic acid) is plasticized using diethylhexyl adipate, polymeric adipates (polyesters of adipic acid), polyethylene glycols of modest molecular weight, citrates, glucosemonoesters, partial fatty acid esters, poly(1,3-butanediol), acetyl glycerol monolaurate, dibutyl sebacate, poly(hydroxybutyrate), poly(vinylacetate), polysaccharides, polypropylene glycol, poly(ethylene glycol-ran-propylene glycol), dioctyl phthalate, tributyl citrate, adipic acid, thermoplastic starch, citrate esters, poly(ε-caprolactone), poly(butylene succinate), acetyl tri-n-butyl citrate, poly-(methyl methacrylate), poly(3-methyl-1,4-dioxan-2-one), diethyl bishydroxymethylmalonate, triethyl citrate, thermoplastic sago starch, oleic acid, glycerol, lactide monomer, lactic acid oligomers, triacetine, glycerol triacetate, monomethyl ethers of poly(ethylene glycol), dioplex, acetyl tri-ethyl citrate, and sorbitol. As indicated in the scientific literature, bulk-eroding polymers may also have a surface-eroding aspect as well, particularly where the polymer is at least partially hydrophobic.

In at least one preferred embodiment, surface-eroding polymers may be used because in some circumstances bulk-eroding polymers may lose mechanical integrity rapidly and suddenly, leaving behind “chunks” of undegraded fiber debris. In contrast, the biodegradation (i.e., bioerosion) rates of surface-eroding polymers may be more controllable and retain mechanical integrity until nearly all the polymer has eroded. For a surface-eroding polymer, the primary factor that governs the release of the energy in the fiber core is the thickness of the polymer shell. As the polymer shell thins, it is less able to resist release of the mechanical energy of the core. Eventually the shell thins to the point where it can no longer resist the core and the core gradually expands or contracts to release its internal stress. As indicated in the scientific literature, surface-eroding polymers may also have a bulk-eroding aspect as well, particularly where the polymer is at least partially hydrophilic.

In a preferred embodiment, two classes of well-studied polymers display surface erosion properties critical to maintaining mechanical integrity during a gradual, well-tuned degradation process: polyanhydrides and polymers formed by polycondensation reactions. The present application discloses members of both classes. Additional classes of surface-erodible polymers lie within the scope of this disclosure as newly discovered.

In at least one preferred embodiment, the core is comprised of poly(glycerol sebacate) (PGS) because it possesses elastin-like properties and can be easily and tunably stretched (i.e., pre-tensioned). PGS has been previously studied for a variety of applications (e.g., scaffolds for chondrocytes, myocytes, heart grafts, and retinal replacement). It has been found that NIH 3T3 fibroblasts grow nearly 50% faster on PGS than on polylactic-co-glycolic acid (PLGA), and further, a highly vascularized collagen forms around the implant in contrast to the fibrotic collagen that forms around PLGA. Additionally, PGS monomers have been approved for human use by the FDA because they are natural components of the lipid production cycle. Previous approval is advantageous because it will decrease the time to clinic by accelerating the FDA 510k approval process. Millimeter thick PGS samples degrade completely in 7 weeks in Sprauge-Dawley rats.

In at least one preferred embodiment, the shell is comprised of polyanhydride, poly(1,3-bis-(carboxyphenoxy)propane) (PCPP), because it can sustain organ weight similar to PLGA but has a linear degradation rate that is even slower than that of PGS. PCPP copolymers have also been approved by the FDA. Because PCPP will be on the external surface, its degradation rate will govern the first portion of the biodegradation process and the tension release timescale, while the PGS will control the amount of fiber contraction and the time to complete biodegradation. By controlling their respective thicknesses, the net degradation rate of the fiber will be highly tunable to achieve the targeted ½- to 24-month degradation window. Development and tuning of these fibers will lead to fewer complications in the surgical treatment of urinary incontinence and pelvic organ prolapse.

In another preferred embodiment, the shell is comprised of a polymer blend of two or more polymers so that the degradation time can be precisely tuned. For example, a mixture of PGS and PCPP or a mixture of PCPP with another polyanhydride, poly(1,3-bis-(carboxyphenoxy)hexane) (PCPH), may be used to shorten or lengthen the degradation time relative to PCPP alone in a homopolymer melt. The mixture of polymers may be uniform and homogeneous or applied in separate coats to create lamina or gradients in the release rates so that the degradation time scale may be precisely controlled.

In a preferred embodiment, the shell comprises surface eroding polymers including, but not limited to, poly(glycerol sebacate), poly(propane-1,2-diol-sebacate) (PPS), poly(butane-1,3-diol-sebacate) (PBS), poly(butane-2,3-diol-sebacate) (PBS), poly(pentane-2,4-diol-sebacate) (PPS), poly(1,3-bis-(carboxyphenoxy)propane) (PCPP), polyanhydride, poly(1,3-bis-(carboxyphenoxy)hexane) (PCPH), poly[1,6-bis(p-carboxyphenoxy)hexane], poly(sebacic acid) diacetoxy terminated, poly[1,4-bis(hydroxyethyl)terephthalate-alt-ethyloxyphosphate], poly[1,6-bis(p-carboxyphenoxy)hexane)-co-sebacic acid], poly[1,4-bis (hydroxyethyl)terephthalate-alt-ethyloxyphosphate]-co-1,4-bis(hydroxyethyl)terephthalate-co-terephthalate), 1,6-bis(p-carboxyphenoxy)hexane, other biodegradable polymers, and other polyester fibers formed by condensation and polyanhydrides.

In at least one preferred embodiment, the core comprises surface-eroding polymers including, but not limited to, poly(glycerol sebacate), poly(propane-1,2-diol-sebacate) (PPS), poly(butane-1,3-diol-sebacate) (PBS), poly(butane-2,3-diol- sebacate) (PBS), poly(pentane-2,4-diol-sebacate) (PPS), poly(1,3-bis-(carboxyphenoxy)propane) (PCPP), polyanhydride, poly(1,3-bis-(carboxyphenoxy)hexane) (PCPH), poly[1,6-bis(p-carboxyphenoxy)hexane], poly(sebacic acid) diacetoxy terminated, poly[1,4-bis(hydroxyethyl)terephthalate-alt-ethyloxyphosphate], polyR1,6-bis(p-carboxyphenoxy)hexane)-co-sebacic acid], poly[1,4-bis(hydroxyethyl)terephthalate-alt-ethyloxyphosphate]-co-1,4-bis(hydroxyethyl)terephthalate-co-terephthalate), 1,6-bis(p-carboxyphenoxy)hexane, other biodegradable polymers, and other polyester fibers formed by condensation and polyanhydrides. In a preferred embodiment, the core is biodegradable, bioerodible, degradable, erodible, photooxidable, or/and photodegradable.

In a preferred embodiment, the core is comprised of non-biodegradable materials including, but not limited to, poly(dimethyl siloxane) (PDMS) (including, for example, silastic MDX4-4210 or MED-4210, inter alia), PDMS with silica (such as bionate 75A, bionate 2, bionate 75D and carbosil 80A, inter alia), polyisoprene, polyethylene oxide, and polyurethane. In a preferred embodiment, the core consists of polymer having linear elastic stress-strain curves.

In at least one preferred embodiment, the shell may have a thickness that varies along the fiber length. In various preferred embodiments, the fiber comprises a shell of uniform thickness, smoothly varying thickness, linearly increasing thickness, sinusoidally varying thickness, sigmoidally increasing thickness, exponentially increasing thickness or summations/combinations thereof. In a preferred embodiment, the shell comprises at least 10% of the fiber diameter. In a preferred embodiment, the fiber comprises a shell of two or more lamina. In a preferred embodiment, the fiber comprises a shell of a continuous gradient of material. In this manner, certain portions of the fiber may release their tension before other sections of the same fiber to apply the contraction more gradually or in a more targeted fashion. In a correlated embodiment, the core may be thicker where the shell is thin to balance the overall degradation time frame for the complete fiber.

In at least one preferred embodiment, the tips of the fibers will be annealed to eliminate preferred planes and interfaces of slippage to prevent delamination of the core from the shell under applied stresses. Should the interface not be completely eliminated, annealing will form a roughened interface to increase the effective Griffiths surface energy parameter, thereby decreasing the potential for delamination at finite tensions.

In another preferred embodiment, the tips of the fibers will be tied or clamped to prevent delamination. For example, the ends of the fibers may be tied in miniature knots.

In at least one preferred embodiment, the shell will comprise at least 10% of the diameter prior to implantation to prevent delamination. Thicker shells are preferred. The shell thickness will control the degradation rate. Multiple shell layers of modest thickness can be stacked to precisely control the degradation rate in vivo.

In at least one preferred embodiment, the elastic modulus of the shell will exceed the elastic modulus of the core by approximately one order of magnitude. Differences of two to four orders of magnitude are fully plausible.

In at least one preferred embodiment, the pretensioned or precompressed fibers are woven, knitted, threaded, or otherwise formed, fabricated, or assembled into a mesh. In a preferred embodiment, the shell is applied to the core-shell fibers by dip coating, evaporative deposition, oxidation of the core (e.g., for PDMS), glued with cyanoacrylates, extruded through a die, polymerization on surface at room temperature, enzymatic polymerization, inter alia. In this or another preferred embodiment, the mesh is implanted within the body to treat urinary incontinence and pelvic organ prolapse and for other plastic and reconstructive surgery applications for other parts or portions of the body. Following implantation, the shell erodes by hydrolysis, enzymatic digestion, bioerosion, or other means, gradually releasing the tension or compression stored in the core. Control over the core and shell material and geometric properties gives the timing, magnitude, and placement of the tension or compression applied to the adjacent tissue.

Even though tissue support comprising pretensioned fibers may initially seem loose and lacking tension at the time of the repair, the gradual contraction of the mesh over time allows the overlying vaginal mucosa and underlying attached pelvic fascia time to accommodate and remodel the new tissue support to reduct the prolapse. The approach of gradually integrating endopelvic fascial support allows for optimal healing and repair without the need to abruptly apply tension to, and potentially over-contract, the endopelvic fascial support, as is the case with the current state-of-the-art pelvic prolapse surgery using natural tissue or mesh augmentation.

In at least one preferred embodiment, the mesh is comprised of N longitudinal fibers where N≧mₜg/(2πE_{c}h²), where mₜ is the mass of the tissue, g is the gravitational constant, E_{c} is the elastic modulus of the core, and h is the radius of the fiber.

In at least one preferred embodiment, the mesh is composed of fibers that expand upon degradation of the fiber shell. This may be advantageous because excessive contraction caused by the formation of collagen or new collagen fibers, along with the infiltration and ratcheting effects of fibroblasts and/or myofibroblasts, is undesirable and may lead to the formation of an avascular collagenous shell. A modest amount of collagen fiber formation and ratcheting is a natural, normal, and necessary part of the healing process. However, when taken to excess, the induced tension can lead to undesirable complications for patients experiencing pelvic floor surgeries. The pre-compressed fibers disclosed herein provide a means of opposing the ratcheting effect and formation of avascular collagen.

In at least one preferred embodiment, the mesh may be comprised of two or more distinct types of fibers having different release times to precisely tune the overall degradation rate of the mesh. This is a biomimetic feature of the present disclosure. The in vivo extracellular matrix dynamically rearranges in response to internal and external stimuli. For example, in wound healing following an inflammatory phase, fibroblasts and/or myofibroblasts infiltrate the wound 1 to 4 days following initial injury, deposit type III collagen, and shrink the wound perimeter. Contraction proceeds at experimentally determined rates of up to 0.75 mm/day, typically peaks at 2 weeks, and can continue, albeit gradually, for months. Models of the interaction between fibroblast and myofibroblast in-migration and wound contraction find both theoretically and experimentally that contraction profiles are, at least partially, sigmoidal. Wound contraction may expedite the healing process by decreasing the amount of granulation tissue and extracellular matrix required to significantly reduce the healing time. Despite the importance of wound contraction to patient healing, synthetic bandages, sutures and surgical implants do not incorporate this important feature. The present disclosure herein enables the design of active surgical mesh that dynamically and controllably contracts or expands to reshape its local environment.

In at least one preferred embodiment, the arrangement, populations and characteristics of the pretensioned fibers within the mesh are comprised in such a manner as to achieve a sigmoidal contraction profile. In a preferred embodiment, this may be achieved by including smaller fibers that erode or degrade quickly with larger and thicker ones eroding slower and more gradually. Alternatively, fibers of the same net diameter but varying shell thicknesses can be arranged so that a few have thin shells, most have intermediate shell thicknesses, and a few have relatively thick shells so as to achieve a sigmoidal contraction profile. Indeed, a wide variety of compositions remain available to achieve sigmoidal, linear, or other contraction profiles. In this manner, additional tension can be preprogrammed into the fibers to gradually contract the patient's fascia, providing increasing levels of support to millions of elderly women, before the fiber biodegrades to avoid mesh erosion. In a preferred embodiment, the fibers and mesh discussed above can be integrated into a sling for treatment of urinary incontinence and pelvic organ prolapse.

In various preferred embodiments, a mesh comprised of fibers disclosed herein contracts or expands by approximately 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm.

In various preferred embodiments, the mesh contracts or expands by approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95%, 100%, 150%, 200%, 250%, or 300%.

In various preferred embodiments, the mesh provides a lift of approximately 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm. In other preferred embodiments, the mesh may provide a lift up to 100 mm or up to 150 mm.

In various preferred embodiments, the fiber mesh and the various fibers therein are tuned to release at an average of up to or approximately 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 70 days, 80 days, 90 days, 100 days, 110 days, 120 days, 130 days, 140 days, or 150 days to gradually contract adjacent tissue.

In at least one preferred embodiment, a preferred contraction or expansion direction is determined, along which a first set of fibers are oriented. A second preferred direction of expansion, contraction, or not expansion/contraction is determined, along which a second set of fibers are oriented. The two directions may or may not be the same. Additional preferred directions for fiber orientation may be determined. Each set of fibers with their respective directional orientations are integrated within a single mesh fabric. In a preferred example, the angle between two preferred directions is 90 degrees. In another preferred example, the angle between two preferred directions is 60 degrees.

In at least one preferred embodiment, the mesh or the core-shell fibers are elements in bandages, and may provide a linear mesh for linear wound healing. Bandages that gradually contract across surface wounds due to blast or burn injury are needed. In particularly severe cases, conventional bandages either have to be removed, perhaps reinjuring and dislodging freshly-adhered cells critical for recovery, or sequentially tightened to control edema. A mesh that allows for a swollen inflammatory phase but then gradually and controllably contracts across the site of injury may lead to improved patient outcomes by minimizing interaction with the wound site and decreasing the nursing monitoring load.

The fibers and the meshes, slings, bandages, tissue scaffolds, and the like, formed from these fibers as disclosed above, have one or more of at least the following five advantages. First, the proposed core-shell fibers will provide previously unprecedented control over the degree of support provided by mesh slings. By refining the fiber dimensions and the ratio of the shell-to-core thicknesses, the fiber tension can be highly tuned. Tension applied by the fiber mesh provides essential support to the tissue. Previously, surgeons have been limited to only meshes composed of relatively stiff polymers (e.g., PE, PP, nylon). The proposed meshes can be tuned to provide a continuum of support ranging from stiff to gentle because they are composed of both soft elastin-like PGS and stiff collagen-like PCPP. The proposed meshes extend a paradigm shift towards softer meshes because they decrease the risk of erosion, urethral stenosis, and voiding dysfunction. Furthermore, a mixture of fiber dimensions and configurations will lead to optimal support necessary to lift the UVJ while providing the softest biocompatible environment possible.

Second, the contraction and/or expansion forces in the proposed core-shell fibers are time released. These are the first timed-release or pre-tensioned fibers designed for reconstructive surgery. Timed release of the tension will minimize the temptation for surgeons to over-tension mesh slings to achieve immediate surgical results, decreasing the risk of tissue erosion and post-surgical voiding dysfunction. The disclosed embodiments draw upon a broad history of timed-release medicines in which coatings are used to precisely tune the rate of drug release (e.g., enteric coating to prevent premature release of aspirin in the stomach to optimize release in the small intestine). Here, the shell coating will precisely time the release of fiber tension, which will translate directly into increased support of the fascia lifting the UVJ, vaginal wall, and pelvic floor. By tuning the fiber dimensions and the amount of fiber pre-tensioning, the tension release rate can be precisely controlled. By combining fibers of different dimensions, the overall tension release rate of the entire mesh can be highly refined. The gradual tensioning of the mesh as it integrates with the attached target tissue would allow the body to accommodate the change in tension and structure in a more natural way than the sudden over-tensioning at the time of the surgery. The disclosure herein provides essential scientific knowledge needed to control the timed-release rate of these novel fibers.

Third, the development of PGS-PCPP core-shell fibers may minimize foreign body responses and enhance tissue regrowth. An increasing body of scientific knowledge suggests that the mechanical properties (e.g., the tensile or shear moduli) of an implant affect the type and density of cells that grow adjacent to it. Relatively stiff meshes have often been encased in thick nonvascularized collagen, leading to overhardened mesh, extensive scar tissue formation, and increased risk of erosion. In contrast, initial studies of the elastin-like PGS showed increased collagen vascularization. Elastin-like polymers provide an enhanced microstress transfer environment, increasing biocompatibility for fibroblasts.

Fourth, by constructing the mesh using specific patterns of various time-released tension fibers, the chronologic and spatial contraction pattern of the mesh can be designed and tailored to meet the structural and functional requirements for plastic and reconstructive surgery in the various body systems.

Fifth, even though the embodiments disclosed herein specifically decrease the patient risk of erosion, urethral stenosis, vaginal stenosis, and voiding dysfunction complications for urinary incontinence and pelvic organ prolapse surgery, the present disclosure has broad implications for all plastic and reconstructive surgeries for all body systems, such as by example, face and neck lifts, breast lifts, body lifts, orthopedic applications and other open or minimally invasive surgeries.

Sixth, by selecting the timing, orientation and extent of contraction or expansion, the mesh is designed such that medical professionals, from the most skilled to the most modestly skilled in the art, may be able to successfully implement the mesh to achieve desired results.

Seventh, the ideal mesh provides mechanical integrity for constructive remodeling to take place, with the help of the extracellular matrix, and then degrades. In this manner, the functionality of the repaired area is preserved, which is not currently the case with vaginal repair mesh. The disclosed degradable shell concept takes important steps toward this objective. The softer core materials, biodegradable or otherwise, provide a more natural feel to the repaired site to avoid erosion.

Furthermore, these core-shell fibers may be incorporated into sutures or suture materials. In at least one preferred embodiment, individual core-shell monofilament fibers comprise the suture. In a preferred embodiment, the individual core-shell monofilament fibers are connected to a needle. In another preferred embodiment, an assembly or a collection of core-shell fibers woven or arranged into a polyfilament fiber comprise the suture. In a preferred embodiment, the polyfilament suture is connected to a needle. In a preferred embodiment, the threads that comprise the polyfilament suture comprise two or more types of fibers that may differ in geometry of material properties. In a preferred embodiment, the fibers in the polyfilament suture are selected to provide a sigmoidal or quasi-sigmoidal contraction profile.

### EXAMPLE 1

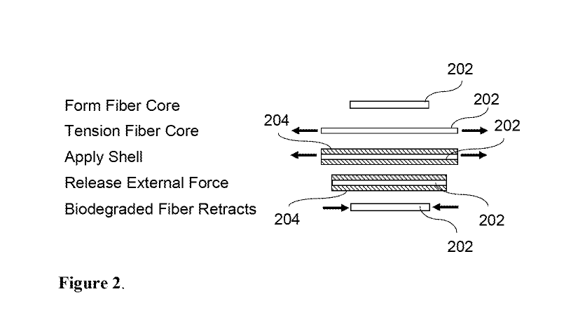

In this example, the equations of linear elasticity are used to model the stress transfer process in three distinct steps (see FIG. 2). First, a cylindrical fiber 202 is stretched from an unstressed state to an applied strain of u_{zz}^{co}. This strain is secured by coating the fiber 202 with an unstressed polymeric shell 204. Second, the applied tension to the core is released, causing it to partially retract and the shell to partially compress. However, the core 202 remains in tension because the shell 204 resists compression via shear forces at the core-shell interface. It is the tension remaining at the end of this step that is available to support the pelvic organs following reconstructive surgery, but excessive tension may also lead to core-shell delamination. Third, the fibers are implanted as a mesh to support organ weight. As the shell biodegrades or bioerodes, the tension stored in the core 202 is released to provide additional lift. The organ weight, along with fiber material properties, determines the effective lift provided by the mesh and the minimum amount of pretensioning required.

Initial Core Tension

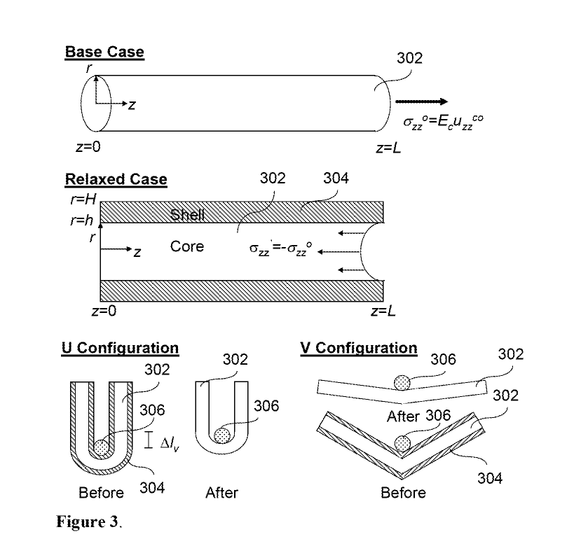

This example considers a core-shell fiber with z oriented along the length of the fiber, r oriented along the radius, and θ in the angular direction (see FIG. 3). A stress σ_{zz}^{o} is applied to the fiber core 302 to induce an increase in length of u_{zz}^{co} and provide the initial tension. This example adopts the notation given by Landau, et al., where the duplicate subscripts denote normal stresses and the superscript “o” represents the initial stress applied to the fiber. The shell 304 of the fiber is then applied in a stress-free manner (e.g., by dip coating, etc.). Because the system is considered to be axisymmetric, this example neglects the angular strains and derivatives thereof. Using the isothermal equations of linear elasticity at steady state in the absence of bulk forces and reducing our domain to stresses below the yield stress (an obvious limit to the effective amount of stress that can be applied), this example determines the following initial stresses and strains in the fiber before it partially retracts. In both the core and the shell, σᵣᵣ^{o}=u_{rz}^{o}=u_{zr}^{o}=σ_{rz}^{o}=σ_{zr}^{o}=σ_{θθ}^{o}=0, in the shell, σ_{zz}^{o}=u_{zz}^{o}=uᵣᵣ^{o}u_{θθ}^{o}=0, and in the core, σ_{zz}^{o}=E_{c}u_{zz}^{co}, u_{zz}^{o}=u_{zz}^{co}, uᵣᵣ^{o}=−v_{c}u_{zz}^{co}, and u_{θθ}^{o}=−v_{c}u_{zz}^{co}. The value u_{zz}^{co} designates the initial strain in the core that provides a mathematical driving force. The elastic moduli in the core and shell are E_{c} and Eₛ, respectively, while the Poisson's ratios in the core and shell are v_{c} and vₛ. The cylindrical coordinates introduce a nonzero u_{θθ}^{o} even though σ_{θθ}^{o} remains zero through the remainder of the analysis.

After Release of Tension

When the clamps that tension the core 302 are released, it retracts partially, developing internal stresses in the absence of surface forces acting on the fiber shell. The core 302 remains held in tension by shear resistance from the shell 304, while the shell 304 compresses as a result of shear stresses from the core 302. Linearity allows us to write

σᵢⱼ=σᵢⱼ^{o}+σᵢⱼ′ and uᵢⱼ=uᵢⱼ^{o}+uᵢⱼ′, (1)

where the prime denotes the additional stress and strain fields developed after release of the external surface force acting on the core alone. Steady state conservation of momentum in the absence of bulk forces demands that

$$\frac{1}{r} \frac{\partial r {σ}_{rr}}{\partial r} + \frac{\partial {σ}_{rz}}{\partial z} = 0 and \frac{1}{r} \frac{\partial r {σ}_{zr}}{\partial r} + \frac{\partial {σ}_{zz}}{\partial z} = 0 \qquad ( 2 )$$

in the isotropic homogeneous media of the core and shell, respectively, with

$${σ}_{rr} = \frac{E}{( 1 + v ) ( 1 - 2 v )} [ ( 1 - v ) {u}_{rr} + {vu}_{zz} + {vu}_{θθ} ] \\ {σ}_{rz} = \frac{E}{( 1 + v )} {u}_{rz} \\ {σ}_{zz} = \frac{E}{( 1 + v ) ( 1 - 2 v )} [ ( 1 - v ) {u}_{zz} + {vu}_{rr} + {vu}_{θ θ} ] \\ {σ}_{zr} = \frac{E}{( 1 + v )} {u}_{zr} \\ {u}_{rr} = \frac{\partial {u}_{r}}{\partial r} \\ {u}_{zz} = \frac{\partial {u}_{z}}{\partial z} \\ {u}_{rz} = \frac{1}{2} ( \frac{\partial {u}_{r}}{\partial z} + \frac{\partial {u}_{z}}{\partial r} ) . \qquad ( 3 )$$

This example employs the well developed assumption such that uᵣ′=uᵣ′(r)≠uᵣ′(z). This, along with the fact that the initial core tension solutions (i.e., the base case) are independent of spatial coordinates, allows for partial decoupling of the equations and the use of separation-of-variables solution.

The decoupled equations are:

$$\begin{matrix} In the core \text{:} In the shell \text{:} & \\ \begin{matrix}\begin{matrix}\frac{1}{r} \frac{\partial }{\partial r} r \frac{\partial {u}_{z}}{\partial z} + \frac{2 ( 1 - {v}_{c} )}{1 - 2 {v}_{c}} \frac{{\partial }^{2} {u}_{z}}{\partial {z}^{2}} = 0 & \frac{1}{r} \frac{\partial }{\partial r} r \frac{\partial {u}_{z}}{\partial r} + \frac{2 ( 1 - {v}_{s} )}{1 - 2 {v}_{s}} \frac{{\partial }^{2} {u}_{z}}{\partial {z}^{2}} = 0\end{matrix} \\ \begin{matrix} {u}_{z}^{′} = 0 & @ z = 0 & {u}_{z}^{′} = 0 & @ z = 0\end{matrix} \\ \begin{matrix} \frac{\partial {u}_{z}^{′}}{\partial z} = \frac{\begin{matrix}( 1 + {v}_{c} ) \\ ( 1 - 2 {v}_{c} )\end{matrix}}{1 - {v}_{c}} ( \frac{{σ}_{t}}{{E}_{c}} - {u}_{zz}^{o} ) @ z = L & \frac{\partial {u}_{z}^{′}}{\partial z} = \frac{\begin{matrix}( 1 + {v}_{c} ) \\ ( 1 - 2 {v}_{c} )\end{matrix}}{1 - {v}_{c}} \frac{{σ}_{t}}{{E}_{s}} @ z = L\end{matrix} \\ \begin{matrix} \frac{\partial {u}_{z}^{′}}{\partial r} = 0 & @ r = 0 & \frac{\partial {u}_{z}^{′}}{\partial r} = 0 & @ r = H\end{matrix} \\ \begin{matrix}{{ \frac{{E}_{c}}{1 + {v}_{c}} \frac{\partial {u}_{z}^{′}}{\partial r} \vert}_{core} = \frac{{E}_{s}}{1 + {v}_{s}} \frac{\partial {u}_{z}^{′}}{\partial r} \vert}_{shell} & @ r = H & { {u}_{z}^{′} \vert}_{core} = {u}_{z}^{′} {\vert}_{shell} & @ r = h\end{matrix}\end{matrix} & \begin{matrix}\begin{matrix}\begin{matrix}\begin{matrix}\begin{matrix}\begin{matrix}\begin{matrix}\begin{matrix}\begin{matrix}( 4 ) \\ \end{matrix} \\ ( 5 )\end{matrix} \\ \end{matrix} \\ \end{matrix} \\ ( 6 )\end{matrix} \\ \end{matrix} \\ ( 7 )\end{matrix} \\ \end{matrix} \\ ( 8 )\end{matrix}\end{matrix}$$

The boundary conditions represent a fixed z axis at the center of the fiber, normal stress applied to the end of the fiber (at z=L), shear stresses on the sides of the fiber, and continuity boundary conditions at the core-shell interface, respectively. The fiber radius is H, the core radius is h, and the half length of the fiber is L prior to relaxation. To ensure decoupling (and therefore refine the problem to make it analytically tractable), radial displacements in Equation 6 are neglected, introducing a small error. The magnitude of this error may be estimated by asserting that uₓₓ′=vu_{zz}′ in Cartesian coordinates or uᵣᵣ′+u′_{θθ}≈−vu_{zz}′ in radial coordinates commensurate with the base case. Then only the right hand side of the second boundary condition changes to

$$\frac{\partial {u}_{z}^{′}}{\partial z} = \frac{( 1 + {v}_{c} ) ( 1 - 2 {v}_{c} )}{1 - {v}_{c} - {v}_{c}^{2}} ( \frac{{σ}_{t}}{{E}_{c }} - {u}_{zz}^{o} ) and$$

$$\frac{\partial {u}_{z}^{′}}{\partial z} = \frac{( 1 + {v}_{s} ) ( 1 - 2 {v}_{s} )}{1 - {v}_{s} - {v}_{s}^{2}} \frac{{σ}_{t}}{{E}_{s}} .$$

The correction, therefore, may be fairly modest and neglected hereafter. Equation 6 provides the primary driving force, while Equation 8 couples together the core and shell displacement and shear. Differences in shear stress boundary conditions can cause a jump condition, the magnitude of which depends on the relative material properties of the core and shell.

These equations are scaled to reduce the number of parameters required in the model without loss of rigor. Scaling simply generates a set of dimensionless ratios or dimensionless parameters to fully explore the parameter space using a minimal set of computations (see Table 1 for groups with representative values).

TABLE 1

| | | Minimum | |
|---|---|---|---|
| Dimensionless Group | Nominal Value | Value | Maximum Value |
| H²/L² | 0.0001 | 0.000025 | 1 |
| v_{c} | 0.45 | 0.25 | 0.5 |
| vₛ | 0.33 | 0.25 | 0.5 |
| u_{zz}° | 1 | 0 | 10 |
| Ē = E_{c}/Eₛ | 0.01 | 10⁻⁶ | 1 |
| h/H | 0.9 | 0 | 1 |
| (γₛ + γ_{c} − γ_{sc})/(E_{c}H) | 2 | 10⁻⁹ | 3 |
| mₜg/(2πNE_{c}h²) | 0.158 | 10⁻⁴ | 10⁵ |
| f_{w} | 0 | 0 | 1 |
| (lₐ/lₘ^{co})² | 0.9 | 0 | 1 |

*based on H = 1 mm, L = 10 cm (1 mm to 20 cm), v_{c} = 0.45, vₛ = 0.33, u_{zz}° = 1 (0 to 10), E_{c} = 1 MPa (0.1-10 MPa), Eₛ = 100 MPa (10-10⁴ MPa), h = 0.8 mm (0-1 mm), (γₛ + γ_{c} − γ_{sc}) = 2 J/m² (0.01-10 J/m²), N = 10 (5-100), mₜ = 0.65 kg (0.075-0.65 kg), lₘ^{co} = 10 cm (1 mm to 20 cm), lₐ = 10 cm(1 mm to 20 cm)

This example scales u_{z}, z, and L on L and uₓ, x, h, and H on H using overbars to denote scaled quantities. The scaled equations are solved using a Finite Fourier Transform (FFT). FFT is the equivalent of separation of variables in the form of

$${\overline{u}}_{z} ( \overline{r} , \overline{z} ) = {\sum }_{n = 0}^{∞} {C}_{n} ( \overline{r} ) {φ}_{n} ( \overline{z} ) , \qquad ( 9 )$$

where Cₙ is a spectral coefficient and φₙ is the basis function. The latter is chosen via the form of the boundary conditions with mixed Neumann and Dirichlet conditions to be

θₙ(\tilde{z})=\sqrt{2}Sin[(n+½)]π\tilde{z}]. (10)

The basis function was chosen to be a function of z to avoid issues arising from discontinuities in shear displacement along the core-shell interface. Multiplying ũ_{z}(\tilde{r},\tilde{z}) by the basis function and integrating with respect to \tilde{z} from zero to unity defines

$${θ}_{n} ( \overline{r} ) ≡ {\int }_{\overline{z} = 0}^{\overline{z} = 1} {\overline{u}}_{z} ( \overline{r} , \overline{z} ) {φ}_{n} ( \overline{z} ) d \overline{z} . \qquad ( 11 )$$

Deen shows that θₙ=Cₙ, such that our final solution is given piecewise as

$${\overline{u}}_{z} ( \overline{r} , \overline{z} ) = \{ \begin{matrix}{\sum }_{n = 0}^{∞} {θ}_{n}^{c} ( \overline{r} ) {φ}_{n} ( \overline{z} ) & for \overline{r} \leq \overline{h} \\ {\sum }_{n = 0}^{∞} {θ}_{n}^{s} ( \overline{r} ) {φ}_{n} ( \overline{z} ) & for \overline{r} > \overline{h} .\end{matrix} \qquad ( 12 )$$

This value of ũ_{z}(\tilde{r},\tilde{z}) represents the displacement after release of the applied tension relative to the length of the shell. The resulting length of the shell is lₛ=lₛ^{o}ũ_{z}(\tilde{r},\tilde{z}) and the resulting length of the core is l_{c}=l_{c}^{o}(u_{zz}^{co}+ũ_{z}(\tilde{r},\tilde{z})). Therefore, negative values of ũ_{z}(\tilde{r},\tilde{z}) are appropriate in each case with more negative values in the core as reported below. Analysis shows ten terms in the summation to be sufficient to achieve accuracies within 0.1%.

Delamination

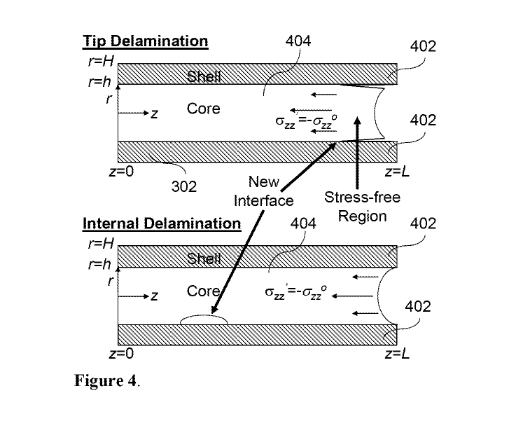

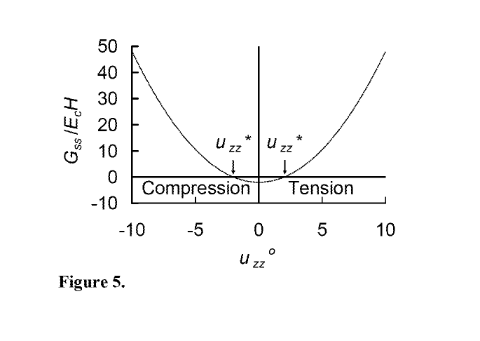

Delamination may occur either at the tip or within the interior (see FIG. 4). However, if delamination occurs in the interior, the core 404 cannot retract and the shell 402 cannot extend to decrease the magnitude of their stress fields, dramatically limiting elastic energy recovery that would drive delamination. FIG. 4 does not show the expansion of the core upon contraction that would self-limit delamination. Therefore, our analysis focuses on delamination from the tip inward whence the core 404 can partially retract and the shell 402 can partially expand to relieve stress. If the energy associated with stress relaxation exceeds the amount of energy required to form the new surface, then the delamination will propagate. Following the pattern given by Griffiths, this example considers a unit area of new crack formation. The new surface energy is

ΔSE=2πhL(γₛ+γ_{c}−γ_{cs}), (13)

where γₛ is the shell-air surface energy, γ_{c} is the core-air surface energy, and γ_{cs} is the core-shell interfacial energy. The interfacial energy can be approximated to first order by γ_{cs}=(γ_{c}γₛ)^{1/2}. The product σᵢⱼuᵢⱼ represents the elastic energy per unit volume. Integrating over the volume of both parts of the fiber gives

$$Δ EE = {\int }_{0}^{2 π} {\int }_{0}^{h} {\int }_{0}^{L} ( {σ}_{ij}^{o} + {σ}_{ij}^{′} ) ( {u}_{ij}^{o} + {u}_{ij}^{′} ) d zr d r d θ + {\int }_{0}^{2 π} {\int }_{h}^{H} {\int }_{0}^{L} ( {σ}_{ij}^{′} ) ( {u}_{ij}^{′} ) d zr d r d θ , \qquad ( 14 )$$

where the first and second terms represent the energy of the core and shell before stress relaxation, respectively. The elastic energy after relaxation is zero because the core and shell are assumed to be stress free. Therefore, the total energy change from before to after crack formation is given as

$$Δ E = - {\int }_{0}^{2 π} {\int }_{0}^{h} {\int }_{0}^{L} ( {σ}_{ij}^{o} + {σ}_{ij}^{′} ) ( {u}_{ij}^{o} + {u}_{ij}^{′} ) d zr d r d θ - {\int }_{0}^{2 π} {\int }_{h}^{H} {\int }_{0}^{L} ( {σ}_{ij}^{′} ) ( {u}_{ij}^{′} ) d zr d r d θ + 2 π hL ( {γ}_{s} + {γ}_{c} - {γ}_{cs} ) . \qquad ( 15 )$$

When ΔE falls below zero, the crack may propagate. This expression may be written in terms of the steady-state energy release rate often reported in the literature on fracture in cylindrical coordinates as Gₛₛ=ΔE/2πhL . Substitution and scaling then yield

$$\frac{{G}_{ss}}{{E}_{c} H} = \frac{1}{2} {u}_{zz}^{{o}^{2}} \overline{h} + \frac{1 - 3 {v}_{c}}{1 - 2 {v}_{c}} \frac{{u}_{zz}^{o}}{\overline{h}} {\sum }_{n = 0}^{∞} \sqrt{2} {( - 1 )}^{n} {\int }_{0}^{\overline{h}} {θ}_{n}^{c} ( \overline{r} ) \overline{r} d \overline{r} + \frac{1 - {v}_{c}}{\overline{h} ( 1 + {v}_{c } ) ( 1 - 2 {v}_{c} )} {\sum }_{n = 0}^{∞} {\int }_{0}^{\overline{h}} {{θ}_{n}^{c} ( \overline{r} )}^{2} \overline{r} d \overline{r} + \frac{{E}_{s}}{{E}_{c }} \frac{1 - {v}_{s}}{\overline{h} ( 1 + {v}_{s} ) ( 1 - 2 {v}_{s} )} {\sum }_{n = 0}^{∞} {\int }_{\overline{h}}^{1} {{θ}_{n}^{s} ( \overline{r} )}^{2} \overline{r} d \overline{r} - \frac{{γ}_{s} + {γ}_{c} - {γ}_{cs}}{{E}_{c} H} \qquad ( 16 )$$

The maximum initial strain, u_{zz}*, that the fiber can sustain without delaminating can be determined from the first law of thermodynamics by setting Gₛₛ=0 leaving u_{zz}*=u_{zz}^{o} as the solution to a quadratic equation with two solutions as shown in FIG. 5. In FIG. 5, the two x-axis intercepts represent u_{zz}* and the values of u_{zz}^{o} between these two intercepts represent strains that will not cause delamination. The positive root is the delamination strain for pretensioned fibers, while the negative root is the delamination strain for precompressed fibers. In other words, the positive root corresponds to delamination from pretensioning, while the negative root corresponds to delamination from precompression.

Tissue Lift After Shell Removal

After the shell 304 has bioeroded, the tension remaining in the core 302 lifts the pelvic organs. This example now determines how much lift the fiber mesh will provide as a function of material properties, initial strain, and weight of the organ 306. For a point source load acting at the ends of the fiber mesh (z=L) such that both halves of each fiber (extending across the entire mesh length) each bear half of the stress, the stress applied by the tissue is

$${σ}_{t} = \frac{{f}_{w} {m}_{t} g}{N 2 π {h}^{2}} , \qquad ( 17 )$$

where mₜ is the mass of the tissue lifted, g is the gravitational constant, and N is the number of fibers each bearing a proportion of the weight. The variable f_{w} is the fraction of the weight sustained by the mesh, where f_{w}=1 if all of the tissue weight is sustained by the fibers, f_{w}=0 if the mesh is loose (e.g., taut but not stretched) or does not bear any of the tissue weight. The vertical lift, Δlᵥ, provided by the freed cores is

Δlᵥ=½(\sqrt{lₘ^{cs}^{²}−lₐ²}−\sqrt{lₘ^{c}^{²}−lₐ²}), (18)

where lₐ is the linear distance between anchor points, lₘ^{cs} is the length of the mesh with core and shell intact (i.e., when inserted and pretensioned), and lₘ^{c} is the length of the mesh after the shell has degraded leaving only the core.

The surgeon places the mesh in either a U shape, such that the two arms of the mesh are nearly parallel, or a V shape, where the two arms of the mesh form a shallow V with respect to each other, as shown in FIG. 3. The selection is determined by which mesh positioning is most appropriate for an individual patient. This example first analyzes the U configuration where the stress (see Equation 17) is related to the strain by

σₜ=E_{c}u_{z}=E_{c}(lₘ^{c}−lₘ^{co})/lₘ^{co}, (19)

where lₘ^{co} represents the initial length of the mesh cores 302. Substituting and solving yields

$$\frac{{l}_{m}^{c}}{{l}_{m}^{co}} = 1 + \frac{{m}_{t} g}{2 π {NE}_{c} {h}^{2 }} . \qquad ( 20 )$$

The last term on the right hand side gives our final dimensionless number which represents the ratio of the stress applied by the tissue to the elastic modulus of the core.

If the two arms form a shallow V, then the solution is more intricate. The force applied by gravity from the tissue 306 to the fiber varies with the angle of the fiber relative to the gravitational vector, ψ. The stress applied by the tissue then becomes

$${σ}_{t} = \frac{{f}_{w} {m}_{t} g}{N 2 π {h}^{2}} Sin [ ψ ] , \qquad ( 21 )$$

where Cos[Ω]=lₐ/lₘ^{c}. A Taylor series expansion about lₐ/lₘ^{c}=1, gives Sin[ArcCos(lₐ/lₘ^{c})≈2^{1/2}(1−lₐ/lₘ^{c})^{1/2}. Substituting Equation 21 into Equation 19 and solving results in a cubic equation

$${( \frac{{l}_{m}^{c}}{{l}_{m}^{co}} )}^{3} - 2 {( \frac{{l}_{m}^{c}}{{l}_{m}^{co}} )}^{2} + [ 1 - 2 {( \frac{{m}_{t} g}{2 π {NE}_{c} {h}^{2}} )}^{2} ] ( \frac{{l}_{m}^{c}}{{l}_{m}^{co}} ) + 2 {( \frac{{m}_{t} g}{2 π {NE}_{c} {h}^{2 }} )}^{2} ( \frac{{l}_{a}}{{l}_{m}^{co}} ) = 0. \qquad ( 22 )$$

The length of the fiber with or without an applied stress is lₘ^{cs}=lₘ^{co}(u_{zz}^{o}+ũ_{z}+1). Therefore, the lift for the U configuration is given by

$$\frac{Δ {l}_{v}}{{l}_{m}^{co}} = \frac{1}{2} ( \sqrt{{( {u}_{zz}^{o} + {\overline{u}}_{z} + 1 )}^{2} - \frac{{l}_{a}^{2}}{{l}_{m}^{{co}^{2}}}} - \sqrt{{( 1 + \frac{{m}_{t} g}{2 π {NE}_{c} {h}^{2 }} )}^{2} - \frac{{l}_{a}^{2}}{{l}_{m}^{{co}^{2}}}} ) . \qquad ( 23 )$$

The implications of this equation are evaluated in detail in the next section. FIG. 3 suggests that Equation 23 is sufficient for both U and V configurations at modest stresses.

Results and Discussion

The model above predicts the fiber length immediately before surgical insertion relative to the initial core length given by lₘ^{cs}/lₘ^{co}=u_{zz}^{o}+ũ_{z}+1, the maximum strain that can be applied before delamination given by u_{zz}*, and the amount of lift given by Δlᵥ/lₘ^{co}. The remainder of this analysis will determine how these depend on the dimensionless parameters summarized in Table 1. These parameters include three length scale ratios, namely, a diameter to length ratio, H²/L²; a dimensionless radial thickness of the core, \tilde{h}=h/H; and a ratio of the distance between anchor points to the initial length of the mesh cores, (lₐ/lₘ^{co})². Other parameters include Poisson's ratios for core and shell, v_{c} and vₛ; the pre-tensioning strain, u_{zz}^{o} the ratio of stress applied by the tissue to the elastic modulus of the core, mₜg/(2πNE_{c}h²); the fraction of that stress realized in the mesh, f_{w}; the ratio of elastic moduli of the core to shell, \tilde{E}=E_{c}/Eₛ; and the ratio of surface energy to elastic energy, (γₛ+γ_{c}−γ_{sc})/(E_{c}H).

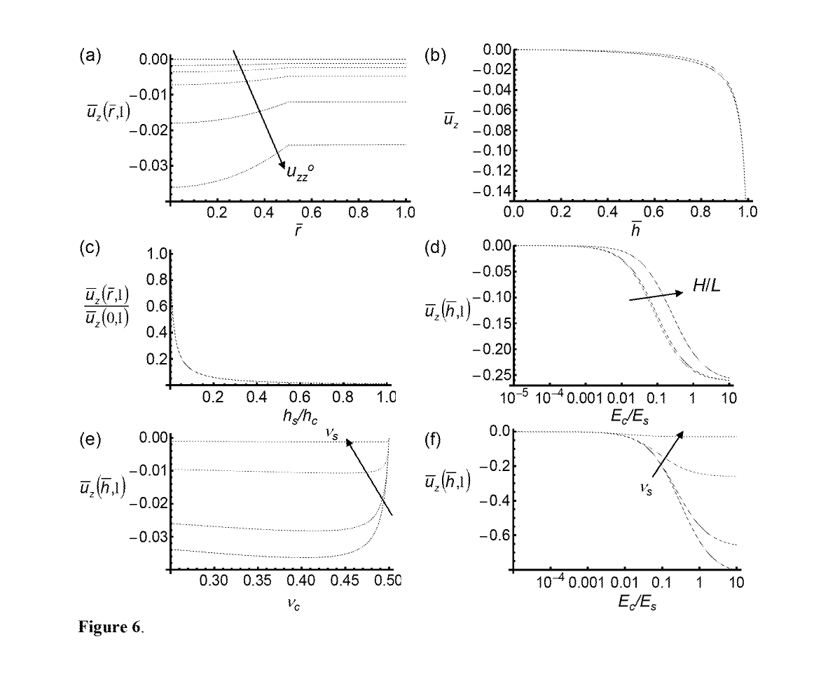

FIG. 6 evaluates the relationship between the preinsertion fiber length (i.e., after fiber production) and these parameters. FIG. 6a shows the deformation profile of the fiber tip. The center of the core experiences most of the deformation as anticipated in FIG. 6, because the shell resists core deformation. For typical initial strains, u_{zz}^{o}, the shell deforms only modestly but compresses significantly as the initial strain, u_{zz}^{o} increases. However, the most influential parameter is the relative thickness of the shell. FIG. 6b shows that the fiber retracts only modestly until the core exceeds 90% of the fiber diameter, at which point the deformation increases dramatically until the fiber retracts completely to its initial prestrained position.

This result is noteworthy because it indicates how, in this example, the fibers will contract in vivo. For bioeroding polymers that degrade steadily, the contraction will be gradual initially but increase steadily as the shell thins linearly. FIG. 6c shows that this is true for all fibers because, regardless of the initial strain, all curves are superimposed. Therefore, it is completely feasible to tune the time of degradation by adjusting the thickness of the shell, because the shell removal rate governs the retraction rate. Furthermore, significant changes in fiber length occur when the shell is ≦10% of the fiber thickness. Mesh design can employ this feature to introduce a delay in tension release by making the shell thickness greater than 1%, 2%, 3%, 4% 5%, 6%, 8%, 10%, 15%, 20%, 25%, 30%, or 35% of the fiber radius.

The remainder of FIG. 6 evaluates the role of the fiber elastic properties. FIG. 6d shows that while the elastic modulus of the shell is two orders of magnitude greater than the modulus of the core, the deformation is fairly modest, but as the two moduli approach parity, the shell cannot resist the shear stress imparted by the core. Therefore, the material with the larger elastic modulus should be on the exterior of the fiber to retain the core tension for subsequent release. FIG. 6e shows that Poisson's ratios only modestly affect deformation, except when either material becomes rubber (i.e., where v_{c} or vₛ approach ½). Indeed, Poisson's ratios of ½ can be used to prevent deformation even when E_{c}/Eₛ increased past unity (see FIG. 6f).

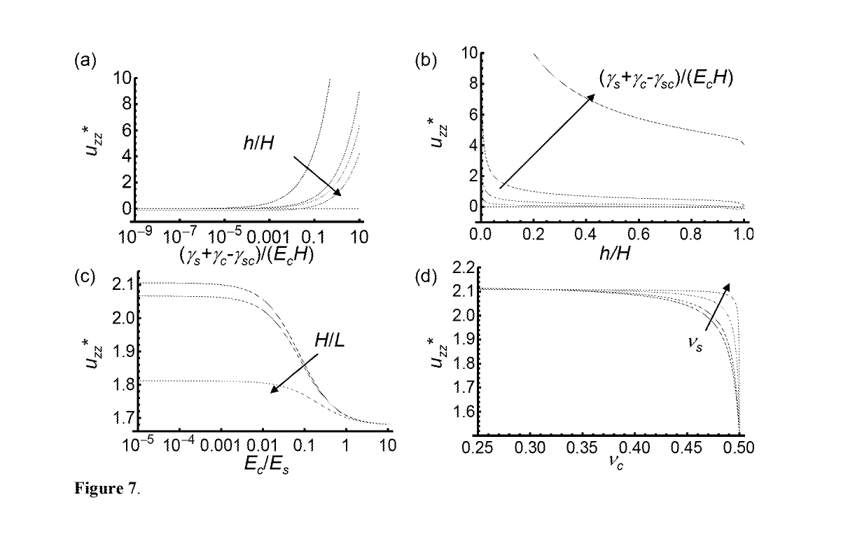

The model also conservatively predicts when delamination may become problematic as seen in FIG. 7. The first two panels show (γₛ+γ_{c}−γ_{sc})/(E_{c}H) and h/H to be the most important parameters in predicting the critical strain for delamination, u_{zz}*. The figure indicates that (γₛ+γ_{c}−γₛ)/(E_{c}H) must be at least 0.01 to allow the core length to double u_{zz}*=1) without delamination. To achieve this level, E_{c} should be fairly modest and perhaps elastin like so that E_{c}=0.1-1.0 MPa and H should also be on the smaller end of the feasible range between 10 nm and 1 mm. However, because each fiber would be smaller, more fibers will be necessary to sustain the same total organ load. The alternative is to adjust the surface energy created by delamination. Surface energies range over several orders of magnitude reflecting numerous sources including van der Waals forces (0.01-0.05 J/m²), cleavage of chemical carbon-carbon bonds (0.4-1.2 J/m²), and also other lumped effects including plasticity. Fracture data from Strobl inclusive of plasticity effects for polystyrene suggests values of 34.3-49.7 J/m². Thus, surface energies span a range exceeding three orders of magnitude, and the exact surface energies for a particular polymer cannot be determined a priori due to the lack of comprehensive tables in the literature but must be measured experimentally for each polymer under consideration at the temperature of use. Notably the formalism developed herein may provide an innovative means of estimating the surface energy by measuring the stress at which core-shell fiber delamination occurs.

However, the pretensioning of the core is inherently a protective measure. As the tension is released, the core expands as required by Poisson's ratio. This expansion will decrease the gap between the core and shell effectively hindering crack propagation. Therefore, one of the key advantages of the pretensioning strategy is that delamination will require a higher initial stress than calculated herein.

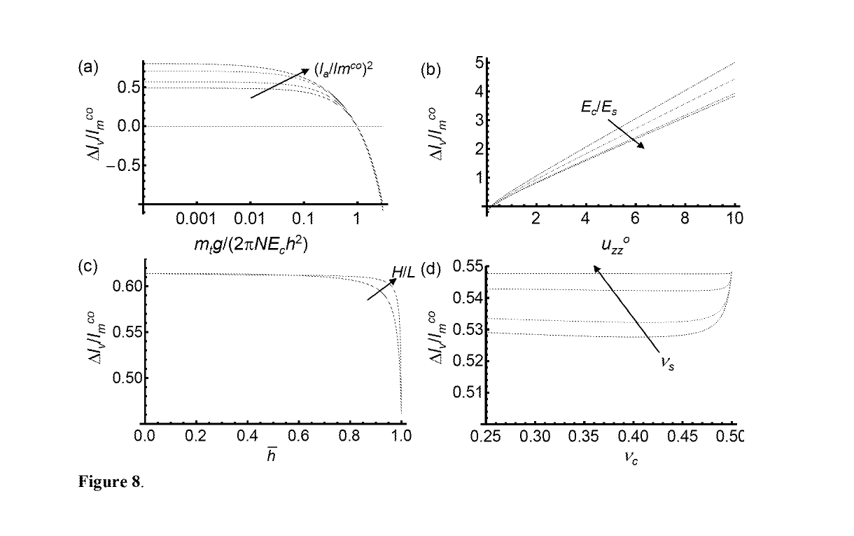

Finally, FIG. 8 shows that the lift, Alₚ, provided by the fibers depends most strongly on mₜg/(2πNE_{c}h²) and u_{zz}^{o}. Only when mₜg/(2πNE_{c}h²)<1 does mesh provide lift to the tissue. Because the tissue weight is set by physiological constraints and varies from patient to patient, any one of three fiber parameters can be used to optimize surgical practice including the elastic modulus of the core, the diameter of the fiber core, and the number of fibers (see FIG. 8a). Each of these variables individually contributes to the strength of the mesh and any of the three can be optimized in practice. FIG. 8b shows that as u_{zz}^{o} increases, the amount of lift increases proportionately. Although delamination provides a limit to the amount of pretensioning that is allowed, it does not significantly curtail the applications of the fibers. As seen in FIG. 7, u_{zz}* can readily achieve values of 2 or more in typical situations leading to values of Δlᵥ/lₘ^{co} as high as unity. An initial core length of 6 cm stretched to 18 cm (for u_{zz}^{o}=2), Δlᵥ/lₘ^{co}=1 translates into a lift of 6 cm, which is more than sufficient for typical clinical scenarios.

The remaining parameters plotted in FIG. 8 play only a minor role in the amount of lift provided. So long as h/H remains less than 0.9, it does not affect the lift (though the lift falls of asymptotically as h/H approaches unity). FIG. 8d shows that more rubbery materials increase the amount of lift because they store more elastic energy than do less rubbery materials. Nevertheless, these parameters only impact the amount of lift at the margins.

In conclusion, this modeling effort indicates the feasibility of pretensioning core-shell fibers to tunably lift pelvic organs. The timing of the lift can be set by adjusting the thickness of the shell, because the shell removal rate governs the retraction rate. Furthermore, because significant changes in fiber length occur when the shell is ≦10% of the fiber thickness, mesh design can introduce a preset delay in deploying the tension release. Delamination considerations limit the amount of pretensioning available, but this is not clinically confining because the fiber core can more than double in length. In net, the load sustained by each fiber must remain approximately below the value of its elastic modulus in order for the fiber to lift the tissue.

### EXAMPLE 2

In this example, proof-of-principle core-shell fibers were generated. The cores were comprised of vulcanized natural rubber bands and the shell coating was comprised of polylactic acid (PLA). To provide tension to the vulcanized natural rubber band, metal cardboard hangers were obtained and the cylindrical cardboard insert removed. The vulcanized natural rubber bands were wrapped around the ends of the hanger. PLA pellets were placed into an uncoated aluminum bread pan and heated to 180-220° C. using an electric hot plate. Higher temperatures decreased the polymer viscosity and formed a more uniform coating. The tensioned vulcanized natural rubber bands were dipped in the PLA melt, withdrawn, allowed to cool, and measured. Coating thicknesses from 1.25-6.00 mm were achieved. In each case, the core-shell rods maintained their lengths following formation of the shell and did not delaminate so long as a coating was applied all the way around the vulcanized natural rubber bands. Partial coatings where one or more surfaces of the vulcanized natural rubber bands remained exposed were susceptible to delamination.

To achieve more uniform coatings, a simple extrusion experiment was performed. A 3.15 mm hole was punctured into the side of the aluminum pan with a screw. The rubber bands were attached to a paperclip and dipped into the PLA. The paperclip was pulled through the hole with the coated vulcanized natural rubber band following it with anchoring or delay to induce a tension stress in this core. Pulling the coated vulcanized natural rubber band through a small hole allowed for the excess PLA (i.e., the shell) to be removed. Thinner more uniform coatings were achieved in two tests of 0.55±0.03 mm (range) and 1.23±0.02 mm (range). These experiments demonstrated that vulcanized natural rubber bands were coated with a thin uniform layer of PLA and that PLA shells were strong enough to fix tension within the stretched vulcanized natural rubber bands nearly three times as thick as the PLA coating.

From the foregoing, it will be appreciated that specific embodiments of the disclosure have been described herein for purposes of illustration, but that various modifications may be made without deviating from the spirit and scope of the disclosure. Aspects described in the context of particular embodiments may be combined with other embodiments or eliminated. Further, although advantages associated with certain embodiments have been described in the context of those embodiments, other embodiments may also exhibit such advantages, and not all embodiments need necessarily exhibit such advantages to fall within the scope of the disclosure.

The embodiments of the invention in which an exclusive property or privilege is claimed are defined as follows:

1. A timed-release tensioned core-shell fiber, comprising:
a shell; and
a core under tension or compression within the shell,
wherein the shell is configured to hold the core under said tension or compression, and
wherein the shell is at least partially removable to release at least a portion of the tension or compression of the core in response to the shell being at least partially removed.

2. The core-shell fiber of claim 1, wherein the shell is removable by erosion or degradation of the shell.

3. The core-shell fiber of claim 2, wherein the erosion or degradation of the shell includes at least one of biodegradation, bioerosion, photooxidation, or photodegradation.

4. The core-shell fiber of claim 2, wherein the erosion or degradation of the shell is controllable.

5. The core-shell fiber of claim 2, wherein the shell comprises a surface-eroding polymer.

6. The core-shell fiber of claim 5, wherein the surface-eroding polymer comprises at least one of a biodegradable polymer, polyester fiber formed by condensation, and polyanhydride.

7. The core-shell fiber of claim 2, wherein the shell comprises a bulk-eroding polymer.

8. The core-shell fiber of claim 1, wherein the shell has a uniform thickness.

9. The core-shell fiber of claim 1, wherein the shell has a varying thickness.

10. The core-shell fiber of claim 9, wherein the shell has at least one of a linearly increasing thickness, a sinusoidally varying thickness, a sigmoidally increasing thickness, or an exponentially increasing thickness.

11. The core-shell fiber of claim 1, wherein the fiber has a diameter, and wherein the shell comprises at least 1%, 2%, 3%, 4%, 5%, 6%, 8%, 10%, 15%, 20%, 25%, 30%, or 35% of the fiber diameter.

12. The core-shell fiber of claim 1, wherein the shell comprises two or more lamina.

13. The core-shell fiber of claim 1, wherein the core comprises a biodegradable, bioerodible, degradable, erodible, photooxidable, and/or photodegradable material.

14. The core-shell fiber of claim 1, wherein the core is a hydrogel core.

15. The core-shell fiber of claim 1, wherein the elastic modulus of the shell exceeds the elastic modulus of the core.

16. The core-shell fiber of claim 1, wherein when the core is under tension, the shell is in compression, and when the core is under compression, the shell is in tension.

17. A fiber mesh comprising core-shell fibers of claim 1.

18. The fiber mesh of claim 17, wherein the core-shell fibers are tuned for timed release of contraction or expansion forces in response to timed release of the tension or compression of the core.

19. The fiber mesh of claim 17, wherein the mesh comprises a first plurality of core-shell fibers of claim 1 having a first orientation or direction, and wherein the mesh comprises a second plurality of fibers having a second orientation or direction.

20. The fiber mesh of claim 19, wherein the second plurality of fibers are comprised of core-shell fibers of claim 1.

21. The fiber mesh of claim 20, wherein the first plurality of core-shell fibers are tuned for timed release of contraction or expansion forces different than the second plurality of core-shell fibers.

22. The fiber mesh of claim 17, wherein the core-shell fibers are configured in multiple orientations or directions, and wherein different core-shell fibers in the fiber mesh are tuned for different timed release of contraction or expansion forces.

23. A medical device, bandage, implant, tissue construct, or sling comprising the fiber mesh of claim 17.

24. The fiber mesh of claim 17, wherein the number of core-shell fibers exceeds mₜg/(2πE_{c}h²), where mₜ is the mass of the tissue, g is the gravitational constant, E, is the elastic modulus of the core, and h is the radius of the fiber.

25. The fiber mesh of claim 17, wherein the mesh is configured using a pattern of time-released core-shell fibers such that the chronologic and spatial contraction pattern of the mesh meets a structural and functional requirement for plastic or reconstructive surgery in a body system.

26. A suture comprising a core-shell fiber of claim 1.
